(19) United States
(12) Patent Application Publication
(10) Pub. No.: US 2007/0282234 A1
(43) Pub. Date: Dec. 6, 2007

(54) TEMPORARY EQUINE COMBINATION WOUND PRESSURE PAD AND BANDAGE

(76) Inventor: Richard RYND, Owings Mills, MD (US)

Correspondence Address: BROOKE SCHUMM III, ONE NORTH CHARLES STREET, SUITE 2450, BALTIMORE, MD, 21201, US

(21) Appl. No.: 11/757,287

(22) Filed: Jun. 1, 2007

Related U.S. Application Data
(60) Provisional application No. 60/803,661, filed on Jun. 1, 2006.

(51) Int. Cl. A61F 15/00

(52) U.S. Cl. 602/41; 602/62; 602/75; 119/850

(57) ABSTRACT

This invention applies direct pressure in such a way as to be effective on a furry and hairy wound, and enable continued mobility by a mammal, especially a horse. The invention also proposes a means of providing a tougher protection for the wound while simultaneously dressing it. The preferred mode of invention proposes a rubberized flexible elastic latex pad applied to the wound, and integrating into that latex pad a bandage mechanism that stretches the pad and can be cinched extremely tightly so that the elastic latex pad applies pressure to the surface of the wound by virtue of the tightly cinched bandage and can be cinched to be a tourniquet if desired.

### CONTINUATION DATA

This application claims benefit of U.S. Provisional application No. 60/803,661 filed on Jun. 1, 2006 and an application of this name filed on Jun. 1, 2007 both of which are adopted by reference.

### FIELD OF INVENTION

This relates to an invention for initial aid by a combination would pressure pad and bandage that can function as a tourniquet.

### SUMMARY

This invention applies direct pressure in such a way as to be effective on a furry and hairy wound, and enable continued mobility by a mammal, especially a horse. A horse is given as a sample use, but in all uses a horse is illustrative for any mammal.

The invention also proposes a means of providing a tougher protection for the wound while simultaneously dressing it.

The preferred mode of invention proposes a rubberized flexible elastic latex pad applied to the wound, and integrating onto the edges of that latex pad a bandage mechanism of elastic tubing that can be cinched extremely tightly so that the latex pad is simultaneously stretched and applies pressure to the surface of the wound by virtue of the tightly cinched bandage and the tubing can be cinched to be a tourniquet if desired.

### BACKGROUND

Horses along with many other riding animals and even beasts of burden, often suffer minor wounds while riding. As most humans know, trying to stop the bleeding of skin of a human over a moving and stressed muscle is very difficult. Humans have more intelligence and communication skills than a horse, so a temporary stop and application of the proverbial Band-Aid elastic adhesive furnishes some relief.

For horses, even for those just a few miles away from the final destination, the loss of blood can be extremely significant, leading to debilitating conditions and making a minor trail incident into a significant adverse health event. Horses also tend to not be particularly calm when wounded, which can further aggravate their injuries and can die on the trail by “bleeding out.”

Traditionally mammalian wounds are handled by stopping bleeding by direct pressure, cleaning the wound, applying a dressing, and then using a bandage to hold the dressing in place. For animals where the wound occurs on a surface with hair or fur, this gets more difficult. In humans, if direct pressure does not stop the bleeding, then application to a pressure point for an extremity can be used. That is generally successful absent a major wound. The final alternative for an extremity on a human is a tourniquet.

While common sense and some prior art suggest a tight gauze dressing to dress wounds on a horse, in fact they do not have the capacity to accomplish the necessary relief and stoppage of bleeding needed to address the wound and facilitate stoppage of bleeding and enable the horse to carry on, and, if not promote healing at least not aggravate the wound. The gauze functions poorly on a hairy or furry surface to put enough pressure.

A traditional tourniquet is undesirable or impossible either because of the mass of a horse extremity, or because it so cuts off the blood supply that the muscles of the extremity involved will no longer function for lack of sustenance by needed blood.

This invention then has as its object to apply direct pressure in such a way as to be effective on a furry and hairy wound, and enable continued mobility by the horse. Contrary to a gauze dressing, the dressing is not to be a dressing to permit drying or aeration of the wound, but to function as a temporary pressure bandage to stop or reduce bleeding until sutures can be administered and the horse immobilized as needed.

The invention also proposes a means of providing a tougher protection for the wound while simultaneously dressing it.

### DESCRIPTION OF FIGURES

FIG. 1 shows a mode where a pad, tubing on each side of the pad, and a strip over each tubing, the strip corresponding with the length along the tubing of the pad, are bonded to each respective tubing.

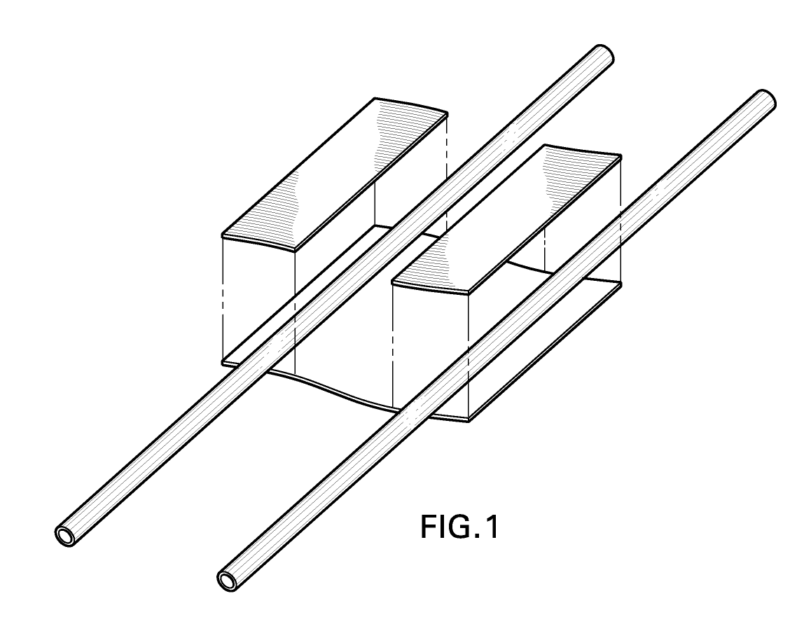

FIG. 2 shows a mode where opposite sides of a pad are wrapped around tubing for each side and bonded to the tubing and back onto the pad.

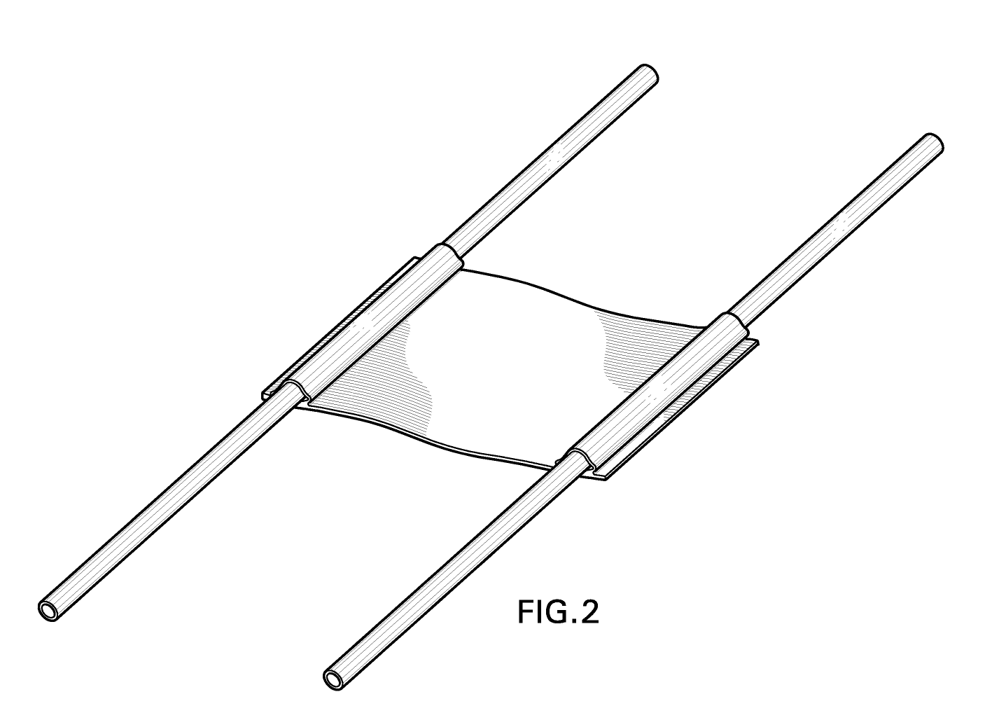

FIG. 3 shows a grommeted variation with tubing running through the grommets.

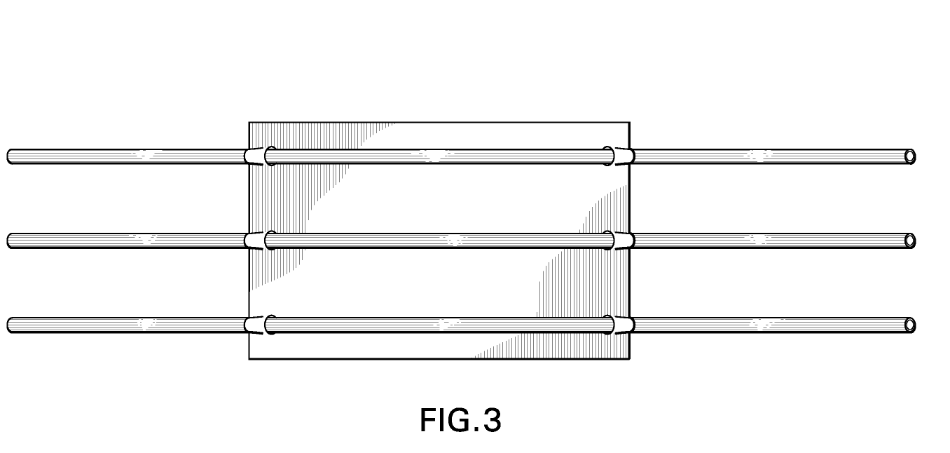

### OBJECTIVES OF THE INVENTION

An object of the invention is to combine the elasticity of a pad with the elastic stretch of tubing to cooperate to increase the perpendicular pressure of a pad on a wound.

An object of the invention is to use a flexible waterproof pad to more effectively stanch bleeding and conform to the surface of the wound.

An object of the invention is to enable the bandage to be used as a tourniquet if necessary.

### DESCRIPTION

This invention therefore contemplates using the combination of an innovative dressing and bandage in order to accomplish the invention.

The preferred mode of invention proposes a rubberized flexible polyethylene latex pad applied to the wound as a dressing, and integrating into that latex pad an elastic tubing bandage mechanism that can be cinched extremely tightly so that the elastic tubing and latex pad cooperating together apply pressure to the surface of the wound by virtue of the tightly cinched bandage. The pad can be any elastic material that is at least water repellent, if not waterproof. The water repellent or waterproof characteristic is defined to be referred to as water repellent. The characteristic of being water repellent protects the wound while acting to stanch the bleeding by application of direct pressure. If tightened further, the tubing can act as a tourniquet.

The invention contemplates several modes. One mode is to integrate grommets into the elastic rubberized pad and pass surgical tubing through them, preferably secured so the tubing will not fall out of the grommets. Two sets of two grommets with a tubing for each set of grommets at each end of the pad are contemplated. A less desirable variation is to simply penetrate the elastic pad with holes to accommodate the tubing or straps instead of using grommets. Securing the tubing to the elastic pad could be done by bonding the tubing to the pad, or by friction, or by having stops. The tubing running through the grommets could have stops to enable the tubing, as it is cinched in place, to stretch the pad. If a horse became wounded, the rubberized pad would be applied directly to the wound, and the tubing tied tightly so the ride could continue. Alternatively, elastic straps, or tubing, tied or tethered by a “VELCRO” hook and felt attachment can be utilized so the horse can ambulate.

Alternatively, the tubing could be integrated into the rubberized pad through apertures through the pad, or by integrated manufacture, to run across the pad and then be longitudinal to the pad in parallel when the pad is mounted on a mammal such as a horse, and surrounds an extremity. If a horse became wounded, the rubberized pad would be applied directly to the wound, and the tubing tied tightly so the ride could continue. A third tubing could be added to apply pressure directly over the wound.

A specific material and mode is to use standard rubber elastic surgical tubing 1 cm. in diameter. Hollow tubing is lighter and more flexible, but not necessary. Two tubings of 65 cm. are cut and placed parallel to each other about 12 cm. apart. An elastic pad is selected that is wide enough to exceed 12 cm. and be bonded to the tubing, and have an edge showing to be bonded to a strip on the opposite side of the tubing. This means a pad for a 1 cm. tubing of approximately 15.5 cm., thereby leaving some 1.75 cm. on each side to surround the tubing and be bonded to the strip to be described. The pad is selected to be 16 cm. in length in the longitudinal direction parallel to the tubings, but any size can be selected. The pad should be made of material that stretches optimally the same amount as the tubing to which it is being bonded when the tubing is stretched. This enables the tubings and pad to best cooperate together to produce the object of stretching to place firm surface pressure on the wound interior to the circumference of the tubings wrapped around a horse's limb and perpendicularly to the pad, and conforming to the wound surface.

The sides of the pad measuring 16 cm. in length are best bonded to the tubing by using an overlap of the pad around the tubing. The 1.75 cm. could be larger to surround the tubing and be bonded back on the pad after curving around the tubing. A separate strip of the pad material can be laid parallel to the tubing so that three layers of pad, tubing and strip are in juxtaposition. The pad and strip can be bonded to the tubing, and the two sides of the strip can be bonded to the pad. This bonding is repeated for the tubing on each side of the pad.

As an example, for application to the limb of a horse or mammal, the first tubing or strap is applied and tightened to hold firmly on the limb. While being tightened the pad and tubing cooperate to stretch the pad. The pad is stretched over the wound and the second tubing is applied firmly while also stretching the pad and holding firmly to the limb. The tubing and pad cooperate to stretch longitudinally as the tubing is stretched and to thereby cooperate with the tubing to facilitate the application of pressure. The tubing can be pulled farther apart from each other to stretch the pad in both the longitudinal and cross-wise directions. By stretching the tubings longitudinally, thereby stretching the pad, and by attaching the tubing farther apart to put tension on the pad perpendicular to the tubing, the tubing and pad cooperate together to place perpendicular pressure on the wound, control bleeding of the wound, encourage clotting inside the water repellent pad, and keep the wound clean.

While the invention is focused on a limb, or extremity including the head and neck, or the tail, the invention can be extension of the proposed tubings, and cinching them to size and cutting off any excess size, can then also be used on the trunk of a mammal to apply significant pressure. Some care must be exercised to not unduly restrict breathing or other body function. No invention exists which demonstrates such versatility as to locating such a direct pressure wound relief on a variety of mammals, especially furry mammals, including a human.

The pad can be any elasticized material. Most flexible elastic polymer materials, rubber or synthetic rubber work well.

Additionally, medicaments such as anti-bacterials, coagulants or salves can be incorporated into or placed on the surface of the rubberized pad.

Additionally gauze could be optionally added to reduce friction and increase comfort, but it may also reduce the effective pressure to stop bleeding.

These and similar embodiments which can be envisioned by a reasonably skilled horseman or reasonably skilled practitioner in the medicinal or veterinary arts which are equivalents are contemplated to be included the invention.

1. A temporary equine combination pressure pad and bandage for a wound of a mammal, comprising:
a water repellent elastic pad capable of being stretched in at least one longitudinal direction;
at least two elastic straps capable of being attached circumferentially around at least part of a mammal;
said at least two elastic straps having sufficient elasticity to be self-securing when attached after application circumferentially around said at least part of a mammal;
said pad being secured on two opposite sides to said at least two elastic straps parallel to said at least one longitudinal direction so that as said at least two elastic straps are attached circumferentially around said at least part of a mammal by stretching said straps, said pad stretches as said at least two elastic straps are stretching, thereby enabling said at lest two elastic straps and said paid to cooperate to apply water-repellent pressure to said wound of said mammal perpendicularly to said pad, interiorly to said circumferential attachment of said straps, and conformably to the surface area of said wound on said mammal, thereby stanching blood flow and occluding foreign materials from said wound.

2. The bandage according to claim 1, further comprising:
gauze interior to said pad to contact said wound.

3. The bandage according to claim 1, further comprising:
a medicament integrated into said gauze.

4. The bandage according to claim 1, further comprising:
a medicament integrated into said pad.

5. The bandage according to claim 1, further comprising:
an additional elastic strap added to enable specific pressure over said elastic pad on said wound.

6. The bandage according to claim 1, further comprising:
at least four grommets adjacent to the corners of said pad; and
said tubing being secured to said pad on each said side of said two opposite sides of said pad.

7. The bandage according to claim 6, further comprising:
gauze interior to said pad to contact said wound.

8. The bandage according to claim 6, further comprising:
a medicament integrated into said gauze.

9. The bandage according to claim 6, further comprising:
a medicament integrated into said pad.

10. The bandage according to claim 6, further comprising:
an additional elastic strap added to enable specific pressure over said elastic pad on said wound.
